(19) United States
(12) Patent Application Publication
(10) Pub. No.: US 2016/0000110 A1
(43) Pub. Date: Jan. 7, 2016

(54) CAFFEINE-REDUCED CACAO COMPOSITION

(71) Applicant: BOURBON CORPORATION, Kashiwazaki-shi, Niigata-ken (JP)

(72) Inventors: Yuto MARUTA, Kashiwazaki-shi (JP); Kinji KOBORI, Kashiwazaki-shi (JP); Shigeru MINEO, Kashiwazaki-shi (JP)

(21) Appl. No.: 14/758,353

(22) PCT Filed: Jul. 23, 2013
(86) PCT No.: PCT/JP2013/069899

§ 371 Date: Jun. 29, 2015

(30) Foreign Application Priority Data
Dec. 28, 2012 (JP) 2012-288854

(51) Int. Cl. A23G 1/30; A23G 1/00; A61K 36/185

(52) U.S. Cl. CPC A23G 1/30; A61K 36/185; A23G 1/0009; A23V 2002/00

(57) ABSTRACT

The present invention relates to a caffeine-reduced cacao composition and a method for producing the same. According to the present invention, it is possible to provide a novel caffeine-reduced cacao composition in which only caffeine is selectively reduced while retaining a health functional component contained in a cacao raw material, and a method for producing the same.

### CROSS-REFERENCE TO RELATED APPLICATIONS

The present application enjoys the benefit of priority of the prior Japanese Patent Application No. 2012-288854, filed on Dec. 28, 2012, the entire content of which disclosures are incorporated herein by reference.

### BACKGROUND OF THE INVENTION

1. Field of the Invention

The present invention relates to a caffeine-reduced cacao composition, and a method for producing the same.

2. Background Art

In recent years, cacao products such as chocolate and cocoa attract attention as not only shikohin (pleasure products), but also foods and drinks which contain a lot of health functional components such as theobromine, polyphenol, and dietary fiber. Meanwhile, cacao products also contain a lot of caffeine.

Caffeine is a component having central nervous stimulating effect as main pharmacological effect and is used in the field of foods and drinks for the purpose of activity promotion, improvement in concentration power due to relieving malaise, or shake off sleepiness, in addition to pharmaceuticals. However, it has been known that caffeine causes palpitations, dizziness, and insomnia as side effects. Particularly, side effects are likely to appear in persons who take a certain specific drug and elderly persons having functional depletion. When an insomniac person intakes, deterioration of symptoms may occur. It is also pointed out that spontaneous abortion and inhibition of fetal development may occur when pregnant women intake caffeine. In order to reduce such health risk due to caffeine, there has been requested to develop cacao products with less caffeine.

In the field of foods and drinks typified by caffeinless coffee, a supercritical fluid extraction method is often used as a method for reducing caffeine. However, this method had a drawback that components other than caffeine are simultaneously extracted and removed from the raw material.

It is known that 71% or more of theobromine resembling caffeine is simultaneously extracted and removed when a supercritical fluid extraction method is actually used using a raw unroasted cacao nib as a raw material (see Japanese Unexamined Patent Publication (Kokai) No. 57-181641 (Patent Literature 1)).

As far as the present inventors know, there was not any report on a cacao composition in which only caffeine is reduced while retaining health functional components such as theobromine and polyphenol.

### CITED LITERATURE

### Patent Literature

[Patent Literature 1]

Japanese Unexamined Patent Publication (Kokai) No. 57-181641

### SUMMARY OF THE INVENTION

The present inventors have now found that, when a specific cacao raw material is subjected to supercritical carbon dioxide extraction using water as a cosolvent, it is possible to obtain a caffeine-reduced cacao composition in which the concentration of theobromine in the composition is 0.7% by weight or more, and the content of theobromine in the composition is 25 times or more of the content of caffeine. Specifically, the present inventors have also found that 60% by weight or more of the content of caffeine derived from the cacao raw material can be reduced while retaining 90% by weight or more of the content of theobromine derived from the cacao raw material. The present inventors have also found that the content of polyphenol in the caffeine-reduced cacao composition is 40 times or more of the content of caffeine and, specifically, 70% by weight or more of the content of polyphenol derived from the cacao raw material is retained. Thus, the present inventors have succeeded in the production of a cacao composition in which only caffeine is selectively reduced without imparting significant change to the composition of theobromine and polyphenol. The present invention has been accomplished based on these findings.

Therefore, an object of the present invention is to provide a novel caffeine-reduced cacao composition in which only caffeine is selectively reduced while retaining a health functional component contained in a cacao raw material, and a method for producing the same. Another object of the present invention is to provide a food and drink composition containing this caffeine-reduced cacao composition.

The caffeine-reduced cacao composition according to the present invention is a composition obtained by subjecting at least one cacao raw material selected from cacao mass and cocoa powder to supercritical carbon dioxide extraction using water as a cosolvent, wherein the concentration of theobromine in the composition is 0.7% by weight or more, and the content of theobromine in the composition is 25 times or more of the content of caffeine.

According to preferred aspect of the present invention, the content of polyphenol in the composition is 40 times or more of the content of caffeine in the above-mentioned composition.

According to preferred aspect of the present invention, the cacao raw material is a cacao raw material which is not subjected to an alkali treatment in the above-mentioned composition.

According to more preferred aspect of the present invention, the cacao raw material is cocoa powder in the above-mentioned composition.

According to an aspect of the present invention, supercritical carbon dioxide extraction is performed under the conditions of 20 MPa or more and 50° C. or higher in the above-mentioned composition.

According to an aspect of the present invention, the proportion of the cosolvent relative to the cacao raw material in supercritical carbon dioxide extraction is 30% by weight or more in the above-mentioned composition.

According to an aspect of the present invention, 90% by weight or more of the content of theobromine derived from the cacao raw material is retained, and also 60% by weight or more of the content of caffeine derived from the cacao raw material is reduced in the above-mentioned composition.

According to preferred aspect of the present invention, 70% by weight of the content of polyphenol derived from the cacao raw material is retained in the above-mentioned composition.

According to another aspect of the present invention, there is provided a food and drink composition comprising the caffeine-reduced cacao composition according to the present invention.

The method for producing a caffeine-reduced cacao composition according to the present invention comprises subjecting at least one cacao raw material selected from cacao mass and cocoa powder to supercritical carbon dioxide extraction using water as a cosolvent to obtain a cacao composition in which theobromine derived from the cacao raw material and, if necessary, polyphenol derived from the cacao raw material is retained, and also caffeine derived from the cacao raw material is reduced.

According to an aspect of the present invention, the concentration of theobromine in the obtained caffeine-reduced cacao composition is 0.7% by weight or more, and the content of theobromine is 25 times or more of the content of caffeine in the above-mentioned method.

According to preferred aspect of the present invention, the content of polyphenol in the obtained caffeine-reduced cacao composition is 40 times or more of the content of caffeine in the above-mentioned method.

According to an aspect of the present invention, supercritical carbon dioxide extraction is performed under the conditions of 20 MPa or more and 50° C. or higher in the above-mentioned method.

According to preferred aspect of the present invention, the proportion of the cosolvent relative to the cacao raw material in supercritical carbon dioxide extraction is 30% by weight or more, and the cacao raw material is humidified by adding the cosolvent, and then supercritical carbon dioxide extraction is performed in the above-mentioned method.

According to preferred aspect of the present invention, static extraction is performed by keeping a pressure-up state in supercritical carbon dioxide extraction in the above-mentioned method.

According to preferred aspect of the present invention, a solvent ratio of the weight of the solvent composed of carbon dioxide to the weight of the cacao raw material is 55.0 or more in the above-mentioned method.

According to an aspect of the present invention, 90% by weight or more of the content of theobromine derived from the cacao raw material in the obtained caffeine-reduced cacao composition is retained, and also 60% by weight or more of the content of caffeine derived from the cacao raw material in the obtained caffeine-reduced cacao composition is reduced in the above-mentioned method.

According to preferred aspect of the present invention, 70% by weight or more of the content of polyphenol derived from the cacao raw material in the obtained caffeine-reduced cacao composition is retained in the above-mentioned method.

The method according to the present invention is a method for retaining the content of theobromine derived from the cacao raw material 90% by weight or more and, if necessary, for retaining the content of polyphenol derived from the cacao raw material 70% by weight or more and also for reducing the content of caffeine derived from the cacao raw material 60% by weight or more, comprising subjecting a cacao raw material to supercritical carbon dioxide extraction using water as a cosolvent.

According to the present invention, it is possible to provide a novel caffeine-reduced cacao composition in which only caffeine is selectively reduced while retaining a health functional component contained in a cacao raw material, and a method for producing the same. In other words, it is possible to provide a caffeine-reduced cacao composition in which only caffeine is selectively reduced without imparting significant change in the composition of health functional components which are originally included in cacao products. The caffeine-reduced cacao composition of the present invention is a composition in which lipid is also reduced by supercritical carbon dioxide extraction. In this way, even persons taking medication, elderly persons, pregnant women, and insomniac persons, who have difficulty in intaking cacao products since there is a need to avoid a risk due to caffeine, can safely intake the caffeine-reduced cacao composition of the present invention for the purpose of promoting health.

### BRIEF DESCRIPTION OF THE DRAWINGS

FIG. 1 is an example of a graph showing a caffeine reduction ratio, and theobromine and polyphenol retention ratios of each cacao raw material (compositions 1 to 3 of Example 1) after a treatment of supercritical carbon dioxide extraction.

FIG. 2 is an example of a graph showing a caffeine reduction ratio, and theobromine and polyphenol retention ratios after a treatment of supercritical carbon dioxide extraction at various temperatures and pressures.

FIG. 3 is an example of a graph showing a procyanidins retention ratio after a treatment of supercritical carbon dioxide extraction at various temperatures and pressures.

FIG. 4 is an example of a graph showing an ORAC value after a treatment of supercritical carbon dioxide extraction at various temperatures and pressures.

FIG. 5 is an example of a graph showing a caffeine reduction ratio, and theobromine and polyphenol retention ratios after a treatment of supercritical carbon dioxide extraction at various properties of a cosolvent.

FIG. 6 is an example of a graph showing a procyanidins retention ratio after a treatment of supercritical carbon dioxide extraction at various proportions of a cosolvent.

FIG. 7 is an example of a graph showing an ORAC value after a treatment of supercritical carbon dioxide extraction at various proportions of a cosolvent.

FIG. 8 shows a regression line of a correlation (r) among the procyanidins concentration and the total polyphenol concentration, and an ORAC value before a treatment of supercritical carbon dioxide extraction (FIG. 8A) and after a treatment of supercritical carbon dioxide extraction (FIG. 8B).

### DETAILED DESCRIPTION OF THE INVENTION

### Caffeine-Reduced Cacao Composition/Production Method

As mentioned above, the caffeine-reduced cacao composition of the present invention is a composition obtained by subjecting a cacao raw material to supercritical carbon dioxide extraction using water as a cosolvent, wherein theobromine derived from the cacao raw material, if necessary, polyphenol derived from the cacao raw material, and, if necessary, procyanidins derived from the cacao raw material are retained (kept, maintained), and caffeine derived from the cacao raw material is reduced.

Here, “caffeine-reduced cacao composition” means a cacao raw material in a state of being modified by subjecting a cacao raw material to a treatment of supercritical carbon dioxide extraction, leading to reduction in caffeine. “Caffeine-reduced” can be used in place of “decaffeinated”, “Decaf (abbreviation of decaffeinated)”, or “caffein less”.

Theobromine is a kind of alkaloids contained in cacao and fall into methylxanthines as caffeine, and also has the same pharmacological effect as that of caffeine. Therefore, theobromine has hitherto been the object to be removed together with caffeine (see Japanese Unexamined Patent Publication (Kokai) No, 57-181641 (Patent Literature 1)). In recent years, theobromine attracts attention as a component having various pharmacological effects such as tranquilizing effect and hypotensive effect (see Physiol Behav. 104 (5); 816-22, 2011).

Polyphenol is a component known as a component having pharmacological effects such as blood glucose level elevation inhibitory effect, anti-stress effect, anti-obesity effect, antioxidation effect, anti-aging effect, anti-caries effect, and anti-angiopathy effect. Examples of polyphenol contained in cacao are typically polyphenol compounds such as procyanidins, quercetin, and tannin. Here, procyanidins mean a polycondensate of catechin or epicatechin.

The caffeine-reduced cacao composition of the present invention is specifically obtained by subjecting a cacao raw material to supercritical carbon dioxide extraction using water as a cosolvent.

Here, “cacao raw material” means a substance which is processed from cacao (Stercurliaceae Theobroma Cacao) beans and serves as an extraction raw material of supercritical carbon dioxide extraction, and examples thereof include cacao beans, cacao nib, cacao mass, cocoa powder, and the like. The cacao raw material used in the present invention contains a lot of theobromine and, if necessary, polyphenol, and cacao mass and cocoa powder are preferred because of satisfactory extraction efficiency of caffeine in supercritical carbon dioxide extraction. These materials may be used alone, or two materials may be used as a mixture.

“Cacao mass” means a substance produced by grinding cacao nib obtained from cacao beans. Cacao mass used in the present invention may be either a solid or a liquid, and there is no particular limitation on state thereof. Here, a liquid of cacao mass is sometimes called a cocoa liquor.

Cacao mass used in the present invention preferably has an average particle diameter of 10 to 200 μm, more preferably 10 to 100 μm, still more preferably 20 to 100 μm, and yet more preferably 20 to 50 μm, from the viewpoint capable of being enhancing extraction efficiency of caffeine. Small particle size leads to a decrease in moving distance from the inside to surface of particles, thus enabling an increase in extraction efficiency. Here, the average particle diameter can be measured using a particle size distribution analyzer with the laser diffraction/scattering method as a measuring principle. For example, the measurement can be performed using a laser diffraction type particle size distribution analyzer SALD-2200 (manufactured by Shimadzu Corporation). In the present invention, “average particle diameter” means a particle diameter at a 50% integrated value in the results of particle size distribution of a dispersion measured using a particle size distribution analyzer with the laser diffraction/scattering method as a measuring principle. Specifically, the number of particles (number) is sequentially added in the increasing order of the particle diameter in particle size distribution, and a diameter of particle when a summed number of particles reaches 50% of the total number of particles means the average particle diameter.

There is no particular limitation on the method of crushing cacao mass into particles having a desired average particle diameter, and it is possible to use a crusher which is usually used in the field of foods and drinks.

“Cocoa powder” means a powder produced by converting cocoa cake, which is obtained by heating cacao mass at 90 to 100° C. and appropriately squeezing cocoa butter as a fat using a pressing machine, into a powder. In the present invention, cocoa cake before crushing is also included in “cocoa powder”. The powder of cocoa powder used in the present invention preferably has an average particle diameter of 10 to 200 μm, more preferably 10 to 100 μm, still more preferably 20 to 100 μm, and yet more preferably 20 to 50 μm.

According to preferred aspect of the present invention, the cacao raw material of the present invention is a cacao raw material which is not subjected to an alkali treatment (alkalization), in other words, cacao mass which is not subjected to an alkali treatment, or cocoa powder which is not subjected to an alkali treatment. Here, the alkali treatment is a means to be performed in the Dutch process which is one of methods for producing cocoa powder, and means that neutralization is performed by adding an alkali agent to cacao nib, cacao mass (mainly, cocoa liquor), or cocoa cake in the production process of cocoa powder. Specifically, cacao mass which is not subjected to an alkali treatment, and cocoa powder which is not subjected to an alkali treatment used in the present invention can be obtained by the Broma process including no alkali treatment. Since the total amount of theobromine and polyphenol derived from the cacao raw material decreases when the alkali treatment is performed, it is possible to obtain a caffeine-reduced cacao composition containing a lot of health functional components such as theobromine and polyphenol by using the cacao raw material which is not subjected to an alkali treatment (see Example 1 of Examples).

According to more preferred aspect of the present invention, the cacao raw material used in the present invention is cocoa powder. Specifically, the content of lipid in cocoa powder of the cacao raw material is preferably 23% or less, more preferably 14% or less, and still more preferably 11% or less. Large lipid content of the cacao raw material may lead to deterioration of extraction efficiency of caffeine in a supercritical fluid extraction method. Meanwhile, small lipid content may lead to a decrease in solvent ratio in supercritical carbon dioxide extraction, thus enabling a decrease in treatment time (see Example 1 of Examples). Here, the lipid content can be measured by the Soxhlet method. Cocoa powder used in the present invention can be obtained by squeezing from cacao mass so as to obtain desired lipid content using a pressing machine. If necessary, the obtained cocoa powder may be further subjected to a degreasing treatment which is usually used in the field of foods and drinks, as long as the content of theobromine and polyphenol do not decrease.

According to more preferred aspect of the present invention, the cacao raw material used in the present invention is cocoa powder which is not subjected to an alkali treatment, the lipid content of which is adjusted to 13 to 14% by the Broma process.

“Supercritical carbon dioxide extraction (supercritical fluid extraction, SFE)” means a method in which a desired extraction substance (caffeine in the present invention) is extracted from a raw material with a supercritical fluid. Here, the supercritical fluid refers to a fluid that is in a state of further exceeding a critical point, at which discrimination is not made between a liquid and a gas, after application of the temperature and pressure to a certain substance (for example, carbon dioxide, water). The fluid used in the present invention is desirably carbon dioxide since it reaches the critical point at comparatively low pressure and low temperature. Because of low polarity, a carbon dioxide fluid in a supercritical state enables extraction of a non-polar substance such as lipid.

“Cosolvent” means a solvent capable of increasing the solubility of a desired extraction substance to enhance extraction efficiency by added dropwise to an extraction raw material before bringing it into contact with a supercritical fluid or bringing it into contact with an extraction raw material together with the supercritical fluid when the desired extraction substance is a polar substance. The cosolvent used in the present invention is desirably water since it can selectively enhance extraction efficiency of caffeine.

According to preferred aspect of the present invention, the method of adding the cosolvent is a method in which the cosolvent is added dropwise before bringing it into contact with the supercritical fluid since the cosolvent is used in a small amount, leading to low cost, and more preferably a method in which humidification (wetting) is performed after dropwise addition. Here, humidification means that the entire extraction raw material is impregnated with the cosolvent, followed by mixing and further permeation of the cosolvent at a predetermined temperature for a predetermined time. The humidification time and temperature can be appropriately set by the amount and particle diameter of the extraction raw material. The humidification time is desirably 5 minutes or more, preferably 10 minutes or more, more preferably 10 to 60 minutes, and still more preferably 10 to 30 minutes. The humidification temperature is preferably 50° C. or higher, more preferably 50 to 100° C., still more preferably 70 to 100° C., and yet more preferably 70° C.

According to more preferred aspect of the present invention, the proportion of the cosolvent relative to the cacao raw material in supercritical carbon dioxide extraction in the humidification method is desirably 30% by weight or more, preferably 30 to 100% by weight, more preferably 30 to 90% by weight, still more preferably 30 to 80% by weight, yet more preferably 30 to 60% by weight, particularly preferably 45 to 60% by weight, and most preferably 45% by weight. If 75% by weight or more of water is added as the cosolvent, a lot of theobromine and polyphenol are likely to be extracted, leading to a decrease in retention ratio (see Example 5(2) of Examples). If 75% by weight or more of water is added as the cosolvent, a lot of procyanidins as a kind of polyphenol are likely to be extracted from the cacao raw material, leading to a decrease in retention ratio (see Example 5(2) of Examples). Therefore, in the case of preparing a caffeine-reduced cacao composition retaining (keeping, maintain) a lot of procyanidins, the proportion of the cosolvent relative to the cacao raw material in supercritical carbon dioxide extraction in the humidification method is preferably 30 to 75% by weight, more preferably 30 to 60% by weight, still more preferably 30 to 45% by weight, and most preferably 30 to 40% by weight.

According to preferred aspect of the present invention, the proportion of the cosolvent in supercritical carbon dioxide extraction is 30% by weight or more, and the cacao raw material is humidified by adding the cosolvent, and then supercritical carbon dioxide extraction is performed.

The pressure of supercritical carbon dioxide extraction is desirably 20 MPa or more, preferably 20 to 50 MPa, more preferably 20 to 40 MPa, still more preferably 25 to 40 MPa, yet more preferably 25 to 30 MPa, and particularly preferably 30 MPa. Here, the pressure (MPa) can be measured by a strain gauge type pressure sensor in a supercritical fluid extraction device.

The temperature of supercritical carbon dioxide extraction is desirably 50° C. or higher, preferably 50 to 100° C., more preferably 50 to 90° C., still more preferably 50 to 80° C., yet more preferably 60 to 80° C., particularly preferably 70 to 80° C., and most preferably 70° C. since less decomposition loss of polyphenol in the extraction raw material enables extraction of caffeine at high efficiency.

According to preferred aspect of the present invention, supercritical carbon dioxide extraction is desirably performed under the conditions of the pressure and temperature of 20 MPa or more and 50° C. or higher. Since only caffeine can be selectively extracted and removed while retaining theobromine and polyphenol in a raw material in high concentration, the pressure and temperature are preferably 20 to 50 MPa and 50 to 100° C., more preferably 20 to 40 MPa and 50 to 80° C., still more preferably 25 to 40 MPa and 50 to 80° C., yet more preferably 25 to 40 MPa and 60 to 80° C., further more preferably 25 to 40 MPa and 70 to 80° C., particularly preferably 25 to 30 MPa and 70 to 80° C., and most preferably 30 MPa and 70° C.

Supercritical carbon dioxide extraction may be either a combination of static extraction and dynamic extraction, or dynamic extraction alone. Supercritical carbon dioxide extraction is preferably a combination of static extraction and dynamic extraction in view of high extraction efficiency of caffeine.

The time of supercritical carbon dioxide extraction is desirably 10 minutes or more, preferably 10 to 60 minutes, more preferably 20 to 60 minutes, still more preferably 20 to 40 minutes, and yet more preferably 20 to 30 minutes, in a pressure-up state controlled to desired pressure and temperature.

According to preferred aspect of the present invention, supercritical carbon dioxide extraction includes performing static extraction while retaining a pressure-up state.

According to preferred aspect of the present invention, a solvent ratio of the weight of the solvent composed of carbon dioxide to the weight of the cacao raw material is desirably 55.0 or more, and preferably 94.0 or more. Here, the solvent ratio means the weight of carbon dioxide used per 1 g of the cacao raw material. A person with an ordinary skill in the art can appropriately set the solvent ratio by the amount of the extraction raw material (amount of treatment, bulkiness of sample), lipid content thereof, particle size, carbon dioxide extraction device to be used, capacity and shape (length, diameter). Accordingly, the solvent ratio is desirably 55.0 to 120.0, and preferably 94.0 to 120.0, under the conditions of an extraction tank used in the below-mentioned Examples. In this case, the extraction ratio of 120.0 means a value at which the amount of caffeine to be extracted and removed from the extraction raw material reaches a saturated state. Accordingly, the solvent ratio is not particularly limited even if it is the value exceeding 120.0, and is preferably 120.0 since the cosolvent is used in a smaller amount, leading to lower cost.

By such supercritical carbon dioxide extraction, caffeine is selectively extracted and removed from the cacao raw material as the extraction raw material to obtain the caffeine-reduced residue of the cacao raw material. Thus, there can be obtained a caffeine-reduced cacao composition in which theobromine derived from the cacao raw material is retained in the residue of the cacao raw material, and also caffeine is reduced.

Specifically, according to the aspect of the present invention, regarding the caffeine-reduced cacao composition, the concentration of theobromine in the composition is desirably 0.7% by weight or more, preferably 0.9% by weight or more, more preferably 1.0% by weight or more, still more preferably 1.1% by weight or more, yet more preferably 1.2% by weight or more, further more preferably 1.3% by weight, particularly preferably 1.4% by weight or more, and most preferably 1.5% by weight or more, and also the content of theobromine in the composition is desirably 25 times or more of the content of caffeine, preferably 30 times or more, more preferably 35 times or more, still more preferably 40 times or more, yet more preferably 45 times or more, further more preferably 50 times or more, particularly preferably 55 times or more, and most preferably 60 times or more. It is generally said that the concentration of theobromine of the cacao raw material varies depending on the production center and kind, and the concentration of theobromine is 0.8% by weight or more (see Gene A. Spiller, Caffeine. CRC Press LLC. 1998). It is generally said that the content of theobromine in cacao beans is about 2 to 10 times the content of caffeine. It is also said that the content of theobromine in cacao products such as cocoa and chocolate is about 3 to 11 times the content of caffeine content (Report: Commodity Test Results of Polyphenol-Containing Foods, National Consumer Affairs Center of Japan, http://www.kokusen.go.jp/pdf/n-20000508_{—}1.pdf; Machiko Kasai et al., “Effect of Cacao Bean Origins on the Palatability of Chocolate”, Journal of the Japanese Society for Food Science and Technology, Vol. 54, No. 7, 332-338, 2007; Japanese Examined Patent Publication (Kokoku) No. 8-4458; Hiroyasu Fukuba et al., “Science and Function of Chocolate/Cocoa”, 2004, See IK Co., Ltd.). Using the measurement method mentioned below by the inventors, commercially available general cocoa powder (available from ADM Company) and ultra-degreased cocoa powder containing about 1% lipid (available from Barry Callebaut Inc.) were actually studied. As a result, it has been found that the content of theobromine was respectively about 13 to 16 times the content of caffeine in these cocoa powders.

More specifically, regarding the caffeine-reduced cacao composition, 90% by weight or more of the content of theobromine derived from the cacao raw material is retained, and also 60% by weight or more of the content of caffeine derived from the cacao raw material is reduced.

According to an aspect of the present invention, regarding theobromine contained in the caffeine-reduced cacao composition, it is desired that 90% by weight or more of the content of theobromine derived from the cacao raw material is retained. Preferably 92% by weight or more, more preferably 95% by weight or more, still more preferably 97% by weight or more, yet more preferably 99% by weight or more, and particularly preferably 100% by weight of the content of theobromine is retained.

According to an aspect of the present invention, regarding caffeine contained in the caffeine-reduced cacao composition, it is desired that 60% by weight or more of the content of caffeine is reduced. Preferably 70% by weight or more, more preferably 80% by weight or more, still more preferably 90% by weight or more, yet more preferably 95% by weight or more, and particularly preferably 100% by weight of the content of caffeine is reduced.

According to preferred aspect of the present invention, regarding the caffeine-reduced cacao composition of the present invention, polyphenol derived from the cacao raw material is further retained in the residue of the cacao raw material.

Specifically, according to preferred aspect of the present invention, regarding the caffeine-reduced cacao composition, the content of polyphenol in the composition is desirably 40 times or more of the content of caffeine, preferably 50 times or more, more preferably 65 times or more, still more preferably 80 times or more, yet more preferably 90 times or more, further more preferably 100 times or more, particularly preferably 110 times or more, and most preferably 120 times or more. It is generally said that the content of polyphenol in cacao products such as cocoa and chocolate is about 13 to 34 times the content of caffeine (Report: Commodity Test Results of Polyphenol-Containing Foods, National Consumer Affairs Center of Japan, http://www.kokusen.go.jp/pdf/n-20000508_{—}1.pdf; Machiko Kasai et al., “Effect of Cacao Bean Origins on the Palatability of Chocolate”, Journal of the Japanese Society for Food Science and Technology, Vol. 54, No. 7, 332-338, 2007). Using the measurement method mentioned below by the inventors, commercially available general cocoa powder (available from ADM Company) and ultra-degreased cocoa powder containing about 1% lipid (available from Barry Callebaut Inc.) were actually studied. As a result, it has been found that the content of polyphenol in these cocoa powders was respectively about 16 to 19 times the content of caffeine.

More specifically, regarding the caffeine-reduced cacao composition, 70% by weight or more of the content of polyphenol derived from the cacao raw material is retained.

Regarding polyphenol contained in the caffeine-reduced cacao composition of the present invention, it is desired that 70% by weight or more of the content of polyphenol derived from the cacao raw material is retained. Preferably 75% by weight or more, more preferably 80% by weight or more, and still more preferably 85% by weight or more of the content of polyphenol is retained.

According to another aspect of the present invention, regarding the caffeine-reduced cacao composition of the present invention, procyanidins as a kind of polyphenols derived from the cacao raw material are further retained in the residue of the cacao raw material.

Specifically, according to preferred aspect of the present invention, regarding the caffeine-reduced cacao composition, the content of procyanidins in the composition is desirably 5 times or more of the content of caffeine, preferably 10 times or more, more preferably 15 times or more, and still more preferably 20 times or more of the content of caffeine.

More specifically, according to preferred aspect of the present invention, regarding the caffeine-reduced cacao composition, 40% by weight or more of the content of procyanidins derived from the cacao raw material is retained.

Regarding the content of procyanidins in the caffeine-reduced cacao composition of the present invention, it is desired that 40% by weight or more of the content of procyanidins derived from the cacao raw material is retained. Preferably 50% by weight or more, more preferably 60% by weight or more, still more preferably 75% by weight or more, and yet more preferably 75% by weight or more of the content of procyanidins is retained.

The content of theobromine and the content of caffeine in cacao raw material or the composition can be measured by high-performance liquid chromatography (HPLC). For example, using an internal standard method of HPLC prominence equipped with Inertsil ODS-3 column (manufactured by Shimadzu Corporation) (solvent: 80% phosphate buffer-20% acetonitrile, flow ratio: 1.0 ml, wavelength: 275 nm), each concentration is calculated from an area ratio based on a calibration curve determined in advance, and then the weight of the cacao raw material or composition containing theobromine and caffeine is integrated, thus determining the content of theobromine and the content of caffeine.

The content of polyphenol in the cacao raw material or composition can be determined by calculating the total polyphenol concentration based on a calibration curve determined in advance using the Folin-Ciocalteu method, and integrating the weight of the cacao raw material or composition containing polyphenol.

The content of procyanidins in the cacao raw material or composition can be determined based on a conventional method for measuring procyanidins mentioned in Kelm, M. A. et al., J. Agric. Food. Chem. 2006, 54(5), 1571-1576, Bergmann, W. R. et al., J. Am. Chem. Soc. 1987, 109, 6614-6619, and the like.

The retention ratio, i.e. the residual ratio (% by weight) of theobromine, polyphenol, or procyanidins can be determined by subtracting the reduction ratio (% by weight) by supercritical carbon dioxide extraction from 100.

Here, the reduction ratio exhibits the weight extracted by supercritical carbon dioxide extraction as a change ratio for every component such as theobromine, caffeine, polyphenol, or procyanidins. Specifically, as shown in the equation mentioned below, it is possible to calculate by dividing the reduction amount obtained by reducing (removing) through supercritical carbon dioxide extraction by the content of the component before extraction.

Reduction ratio (% by weight)=(component content before SFE treatment [component concentration before treatment×weight of cacao raw material before treatment]−component content after SFE treatment [component concentration after treatment×weight of cacao raw material after treatment])/component content before SFE treatment×100 Equation

According to an aspect of the present invention, there is provided a method for producing a caffeine-reduced cacao composition, which comprises subjecting at least one cacao raw material selected from cacao mass or cocoa powder to supercritical carbon dioxide extraction using water as a cosolvent to obtain a cacao composition in which theobromine derived from the cacao raw material and, if necessary, polyphenol derived from the cacao raw material is retained, and also caffeine derived from the cacao raw material is reduced.

According to another aspect of the present invention, there is provided a method for retaining the content of theobromine derived from the cacao raw material 90% by weight or more and, if necessary, for retaining the content of polyphenol derived from the cacao raw material 70% by weight or more and for reducing the content of caffeine derived from the cacao raw material 60% by weight or more, comprising subjecting a cacao raw material to supercritical carbon dioxide extraction using water as a cosolvent.

### Functional Composition/Food and Drink Composition and Pharmaceutical Composition

In the caffeine-reduced cacao composition of the present invention, functional components included in cacao are retained and only caffeine is selectively reduced. Accordingly, it is possible to obtain health promotion effect by a lot of theobromine and, if necessary, polyphenol and, if necessary, procyanidins included in cacao. In other words, the caffeine-reduced cacao composition of the present invention can be used as a functional composition.

In an embodiment of the present invention, theobromine is retained in the caffeine-reduced cacao composition of the present invention. Accordingly, the caffeine-reduced cacao composition of the present invention can be used as a composition for tranquilization (tranquilizer) and a composition for blood pressure decrease (hypotensive agent). According to an aspect of the present invention, there is provided use of the caffeine-reduced cacao composition of the present invention for the production of a composition for tranquilization (tranquilizer) and a hypotensive composition (hypotensive agent). There are also provided a method for tranquilizing and a method for decreasing blood pressure, each comprising administering the caffeine-reduced cacao composition of the present invention to the object who must avoid a risk due to intake of caffeine.

According to an embodiment of the present invention, it is confirmed that the caffeine-reduced cacao composition of the present invention retains polyphenol therein. Accordingly, according to an aspect of the present invention, the caffeine-reduced cacao composition of the present invention can be used as an antioxidation composition (antioxidant). According to an aspect of the present invention, there is provided use of the caffeine-reduced cacao composition of the present invention for the production of an antioxidation composition. There is also provided a method for preventing oxidation comprising administering the caffeine-reduced cacao composition of the present invention to the object who must avoid a risk due to intake of caffeine. Surprisingly, the present inventors have found that antioxidation activity of the caffeine-reduced cacao composition of the present invention is not correlated with the concentration of procyanidins which are said to have strong antioxidation effect, but are positively correlated with the total polyphenol concentration (see Example 5(3) of Examples). Accordingly, even a caffeine-reduced cacao composition in which procyanidins are reduced, it is possible to use as an antioxidation composition, if the total polyphenol concentration is retained in the composition.

According to an embodiment of the present invention, it is confirmed that the caffeine-reduced cacao composition of the present invention retains procyanidins therein. Accordingly, it is also possible to use the caffeine-reduced cacao composition of the present invention as an anti-obesity composition (anti-obesity agent), a cancer inhibition composition (anti-cancer agent), and a composition for improving bloodstream (bloodstream improving agent). According to an aspect of the present invention, there is provided use of the caffeine-reduced cacao composition of the present invention for the production of an anti-obesity composition, a cancer inhibition composition, and a composition for improving bloodstream. There are also provided a method for inhibiting obesity, a method for preventing and/or treating cancer, and a method for improving bloodstream, each comprising administering the caffeine-reduced cacao composition of the present invention to the object who must avoid a risk due to intake of caffeine.

According to an aspect of the present invention, there is provided a food and drink composition comprising a caffeine-reduced cacao composition according to the present invention. According to another aspect, there is provided a pharmaceutical composition comprising a caffeine-reduced cacao composition according to the present invention.

In the present invention, the food and drink composition is not particularly limited, as long as it is a composition other than a pharmaceutical composition and is in an orally ingestable form such as a solution, a suspension, an emulsion, a powder, or a solid molded body. Specific examples of the food and drink composition include chocolate, cocoa drink; foods using chocolate, such as chocolate spread, chocolate syrup, chocolate flower paste, and chocolate coating; and raw materials for imparting chocolate taste to baked confectioneries such as cake and cookie, rice confectionery, tablet confectionery, candy, gummy, ice cream, pudding, breads, and the like. Examples of the other food and drink composition include instant foods such as instant noodles, retort foods, canned foods, microwave oven foods, instant soup/miso soup, and freeze-dry foods; drinks such as refreshing drink, fruit juice drink, vegetable drink, soy milk drink, coffee drink, green tea drink, powder drink, concentrated drink, and alcohol drink; wheat flour products such as bread, pasta, noodle, cake mix, and bread crumbs; confectioneries such as candy, caramel, chewing gum, chocolate, cookie, biscuit, cake, pie, snack food, cracker, Japanese confectionery, and dessert confectionery; seasonings such as sauce, tomato processed seasonings, flavor seasonings, cooking mix, tare sauces, dressings, noodle soups, and curry/stew mixes; oils and fats, such as modified fat, butter, margarine, and mayonnaise; dairy products such as milk-based drink, yoghurts, lactic acid bacteria drink, ice creams, and creams; processed agricultural foods such as canned agricultural foods, jams/marmalades, and cereal; and raw materials used for frozen foods.

The food and drink composition also includes those classified into so-called health foods, supplements, foods with health claims (for example, foods with nutrient function claims), foods for specified uses (for example, foods for specified health uses (including reduction of disease risk claim), and foods for patients, modified milk powder for baby, powdered milk for pregnant women and lactating woman, foods for person with aphagia).

In the present invention, the pharmaceutical composition is prepared as an oral formulation or a parenteral formulation in accordance with a usual method, using pharmaceutically acceptable additives in combination. In the case of the oral formulation, it is possible to take forms, for example, solid formulations such as tablets, powders, fine granules, granules, capsules, pills, and sustained-release tablets; and liquid formulations such as solutions, suspensions, and emulsions. In the case of the parenteral formulation, it is possible to take forms, for example, injections and suppositories. From the viewpoint of simplicity, the oral formulation is preferred. Examples of the pharmaceutically acceptable additive include excipients, stabilizers, antiseptics, humectants, emulsifiers, lubricants, sweeteners, colorants, flavors, buffers, antioxidants pH adjustors, and the like.

A person with an ordinary skill in the art can appropriately set the content of a caffeine-reduced cacao composition in the composition such as a food and drink composition or a pharmaceutical composition by the kind and form of the food and drink or the pharmaceutical composition, and the purpose of preventing and improving.

The amount of intake of a food and drink composition or a pharmaceutical composition is appropriately set according to each case in consideration of the form (dosage form as for drug), age, body weight, and sex of human who intakes, purpose of intake, and the like.

### EXAMPLES

The present invention will be described in detail by way of the following Examples, but the present invention is not limited thereto. Unless otherwise specified, cacao mass used in the following Examples is prepared from cacao beans (produced in Republic of Ghana, available from ITOCHU Food Sales and Marketing Co., Ltd.) by a usual method in Bourbon Corporation.

### Example 1

### Caffeine-Reduced Cacao Composition

### (1) Preparation of Caffeine-Reduced Cacao Composition

Using, as a cacao raw material, cacao mass which is not subjected to an alkali treatment (non-alkali-treated cacao mass), alkali-treated cocoa powder (average particle diameter of 44.6 μm, available from ADM Company) and cocoa powder which is not subjected to an alkali treatment (non-alkali-treated cocoa powder), supercritical carbon dioxide extraction was performed. Cacao mass used is powdered (average particle diameter 114.3 μm) cacao mass produced by a crusher. The non-alkali-treated cocoa powder used is powdered (average particle diameter of 32.7 μm) cocoa cake, which is obtained from cacao mass by the Broma process, using Cocoa Mill (YTK3022, manufactured by Yutaka Manufacturing Company).

The composition of a nutrient component of each cacao raw material before SFE treatment is shown in Table 1.

TABLE 1
Composition of nutrient component

| | Non-alkali-treated | Alkali-treated | Non-alkali-treated |
|---|---|---|---|
| | cacao mass | cocoa powder | cocoa powder |
| Energy (kcal) | 656 | 458 | 434 |
| Protein (g) | 14.2 | 22.0 | 27.3 |
| Lipid (g) | 55.1 | 23.0 | 14.1 |
| Carbohydrate (g) | 25.7 | 40.8 | 49.4 |
| Moisture (g) | 1.8 | 5.3 | 3.4 |
| Ash (g) | 3.2 | 8.9 | 5.8 |
| Sodium (mg) | 5 | 29 | 8 |
| Sodium chloride | 0 | 0.1 | 0 |
| equivalent (g) | | | |

Using a supercritical carbon dioxide extraction device (supercritical fluid extraction system SFX1220 (Model 220), manufactured by Teledyne ISCO, Inc.), extraction was respectively performed under the conditions shown in Table 2 mentioned below. Specifically, water was added as a cosolvent to a cacao raw material charged in an extraction tank to thereby humidify the cacao raw material for 10 minutes. During humidification, the temperature was raised until reaching a desired temperature. Subsequently, carbon dioxide (CO₂) was sent into an extraction tank, and the temperature and pressure were adjusted to the desired temperature and pressure, and then supercritical carbon dioxide extraction was performed. The residue after extraction was obtained as a caffeine-reduced cacao composition (compositions 1 to 5).

TABLE 2
Conditions of supercritical carbon dioxide extraction

| | | Weight (g) | | | | | | | | |
|---|---|---|---|---|---|---|---|---|---|---|
| | | Before | | | Cosolvent | Humidification | | Static | Dynamic | Solvent |
| | Cacao raw | SFE | After SFE | Temperature | (% by | time | Pressure | extraction | extraction | ratio |
| | material | treatment | treatment | (° C.) | weight) | (minutes) | (MPa) | (minutes) | (minutes) | S/F |
| Composition 1 | Non-alkali-treated | 0.501 | 0.272 | 70 | 40 | 10 | 30 | 20 | 30 | 111.2 |
| | cacao mass | | | | | | | | | |
| Composition 2 | Alkali-treated | 0.502 | 0.421 | 70 | 30 | 10 | 30 | 20 | 15 | 55.6 |
| | cocoa powder | | | | | | | | | |
| Composition 3 | Non-alkali-treated | 0.503 | 0.413 | 70 | 30 | 10 | 30 | 20 | 15 | 55.6 |
| | cocoa powder | | | | | | | | | |
| Composition 4 | Non-alkali-treated | 0.501 | 0.414 | 50 | 45 | 10 | 20 | 20 | 15 | 55.6 |
| | cocoa powder | | | | | | | | | |
| Composition 5 | Non-alkali-treated | 0.501 | 0.467 | 70 | 60 | 10 | 30 | 20 | 15 | 55.6 |
| | cocoa powder | | | | | | | | | |

### (2) Analysis

Using high-performance liquid chromatography (HPLC prominence (manufactured by Shimadzu Corporation)), the respective compositions thus obtained were subjected to the measurements of the concentration of theobromine and the concentration of caffeine contained in the composition, and also the concentration of polyphenol was measured using the Folin-Ciocalteu method. Regarding the respective compositions before SFE treatment, the concentration of each component was measured in the same manner. Here, the conditions of HPLC are as follows: Inertsil ODS-3 column, 40° C., solvent: 80% phosphate buffer-20% acetonitrile, flow ratio: 1.0 ml, wavelength: 275 nm. The Folin-Ciocalteu method was specifically performed based on Literature: Standard Tables of Food Composition in Japan, 2010, Chapter 3: Materials, Chapter 4: Analytical Manual (Five components such as iodine, polyphenol).

A ratio of the concentration of caffeine to the concentrations of theobromine and polyphenol thus obtained was determined, and the obtained ratio was respectively designated as T/C and P/C.

Furthermore, a reduction ratio of each component by SFE treatment was determined. Here, the reduction ratio was determined by the following equation.

Reduction ratio (% by weight)=(component content before SFE treatment [component concentration before treatment×weight of cacao raw material before treatment]−component content after SFE treatment [component concentration after treatment×weight of cacao raw material after treatment])/component content before SFE treatment×100 Equation

With respect to theobromine and polyphenol, a retention ratio was determined. Here, the retention ratio was determined by the following equation.

Retention ratio (% by weight)=100−reduction ratio Equation

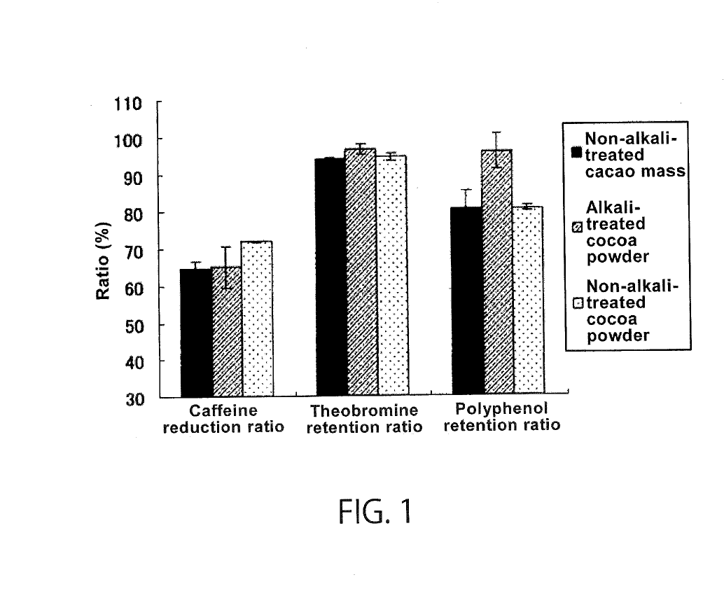

The results are shown in Table 3. With respect to the reduction ratio and retention ratio of compositions 1 to 3, the results are also shown in FIG. 1.

TABLE 3
Component concentration, and reduction ratio/retention ratio, component ratio before and
after supercritical carbon dioxide extraction in each raw material and extraction condition

| | Before SFE treatment | | | | | | | |
|---|---|---|---|---|---|---|---|---|
| | | | | Component | | After SFE treatment | | |
| | Component concentration (mg/g) | | | ratio | | Component concentration (mg/g) | | |
| | Caffeine | Theobromine | Polyphenol | T/C | P/C | Caffeine | Theobromine | Polyphenol |
| Composition 1 | 1.19 ± 0.02 | 15.17 ± 0.03 | 29.05 ± 0.73 | 12.7 | 24.4 | 0.76 ± 0.05 | 25.86 ± 0.30 | 49.72 ± 1.78 |
| Composition 2 | 1.72 ± 0.01 | 23.58 ± 0.32 | 28.79 ± 0.84 | 13.7 | 1.67 | 0.71 ± 0.16 | 26.94 ± 0.32 | 33.11 ± 0.82 |
| Composition 3 | 2.15 ± 0.07 | 29.21 ± 0.13 | 62.32 ± 1.26 | 13.6 | 29.0 | 0.74 ± 0.01 | 33.50 ± 0.28 | 60.97 ± 0.77 |
| Composition 4 | 2.15 ± 0.07 | 29.21 ± 0.13 | 62.32 ± 1.26 | 13.6 | 29.0 | 0.72 ± 0.02 | 33.47 ± 0.34 | 61.19 ± 0.72 |
| Composition 5 | 2.15 ± 0.07 | 29.21 ± 0.13 | 62.32 ± 1.26 | 13.6 | 29.0 | 0.58 ± 0.09 | 33.00 ± 0.51 | 60.21 ± 0.73 |

| | After SFE treatment | | | | |
|---|---|---|---|---|---|
| | | | | Component | |
| | Reduction ratio/Retention ratio (%) | | | ratio | |
| | Caffeine | Theobromine | Polyphenol | T/C | P/C |
| Composition 1 | 64.62 ± 1.83 | 93.98 ± 0.21 | 80.22 ± 5.08 | 34.0 | 65.4 |
| Composition 2 | 64.98 ± 5.68 | 96.50 ± 1.37 | 95.74 ± 4.70 | 37.9 | 46.6 |
| Composition 3 | 71.89 ± 0.15 | 94.32 ± 1.12 | 80.46 ± 0.75 | 45.3 | 82.4 |
| Composition 4 | 69.89 ± 0.15 | 102.51 ± 2.30 | 87.92 ± 3.94 | 46.5 | 85.0 |
| Composition 5 | 75.11 ± 3.66 | 104.52 ± 2.48 | 89.39 ± 1.82 | 56.9 | 103.8 |

A reduction ratio is entered as for caffeine, and a retention ratio is entered as for theobromine and polyphenol.
T/C represents a ratio of theobromine concentration/caffeine concentration, and P/C represents a ratio of polyphenol concentration/caffeine concentration
n = 3, Average ± SE (standard error)

### Example 2

### Food and Drink (Milk Chocolate)

Using caffeine-reduced cacao compositions 1 to 5 obtained in Example 1, and a cacao raw material before SFE treatment of compositions 1 to 3 (non-alkali-treated cacao mass, alkali-treated cocoa powder, non-alkali-treated cocoa powder), milk chocolate was produced according to the formulation shown in Table 4 below.

TABLE 4
Formulation of milk chocolate

| | Mixing ratio |
|---|---|
| Milk chocolate materials | (% by weight) |
| Sugar | 40 |
| Cocoa butter | 19 |
| Cacao raw material before SFE | 9 |
| treatment/caffeine-reduced cacao | |
| compositions 1 to 5 | |
| Vegetable fat and oil | 10 |
| Whole milk powder | 21.3 |
| Emulsifier | 0.6 |
| Flavor | 0.1 |
| Total | 100.0 |

The chocolate thus obtained had no problem in taste and quality and was comparable with commercially available milk chocolate.

According to calculations, chocolate obtained by mixing caffeine-reduced cacao compositions 1 to 5 is chocolate in which the concentration of theobromine in chocolate is at least 0.7% by weight or more, and specifically 2.3 to 3.0% by weight, and the content of theobromine in chocolate is at least 25 times or more of the content of caffeine, and specifically 34 to 63 times (the content of polyphenol in chocolate is at least 40 times or more of the content of caffeine content, and specifically 46 to 115 times).

### Example 3

### Food and Drink (Cocoa Cookie)

Using caffeine-reduced cacao compositions 1 to 5 obtained in Example 1, and a cacao raw material before SFE treatment (non-alkali-treated cacao mass, alkali-treated cocoa powder, non-alkali-treated cocoa powder), cocoa cookie was produced according to the formulation shown in Table 5 below.

TABLE 5
Formulation of cocoa cookie

| | Mixing ratio |
|---|---|
| Cocoa cookie materials | ( % by weight) |
| Wheat flour | 38 |
| Butter | 38 |
| Sugar | 13 |
| Cacao raw material before SFE | 6 |
| treatment/caffeine-reduced cacao | |
| compositions 1 to 5 | |
| Emulsified fat | 3 |
| Whole milk powder | 1 |
| Egg | 1 |
| Total | 100.0 |

The cocoa cookie thus obtained had no problem in taste and quality and was comparable with commercially available cocoa cookie.

According to calculations, cocoa cookie obtained by mixing caffeine-reduced cacao compositions 1 to 5 is cocoa cookie in which the concentration of theobromine in cocoa cookie is at least 0.7% by weight or more, and specifically 1.5 to 2.0% by weight, and the content of theobromine in cocoa cookie is at least 25 times or more of the content of caffeine, and specifically 33 to 63 times (the content of polyphenol in cocoa cookie is at least 40 times or more of the content of caffeine content, and specifically 46 to 117 times).

### Example 4

### Food and Drink (Cocoa Drink)

Using caffeine-reduced cacao compositions 1 to 5 obtained in Example 1, and a cacao raw material before SFE treatment (non-alkali-treated cacao mass, alkali-treated cocoa powder, non-alkali-treated cocoa powder), cocoa drink was produced according to the formulation shown in Table 6 below.

TABLE 6
Formulation of cocoa drink

| | Mixing ratio |
|---|---|
| Cocoa drink (powder) materials | (% by weight) |
| Sugar | 44 |
| Lactose | 15 |
| Cacao raw material before SFE | 20 |
| treatment/caffeine-reduced cacao | |
| compositions 1 to 5 | |
| Whole milk powder | 10.3 |
| Powdered skim milk | 10.3 |
| Salt | 0.1 |
| Emulsifier | 0.3 |
| Total | 100.0 |

The cocoa drink thus obtained had no problem in taste and quality and was comparable with commercially available cocoa drink.

According to calculations, cocoa drink obtained by mixing caffeine-reduced cacao compositions 1 to 5 is cocoa drink in which the concentration of theobromine in cocoa drink is at least 0.7% by weight or more, and specifically 5.1 to 6.7% by weight, and the content of theobromine in cocoa drink is at least 25 times or more of the content of caffeine, and specifically 34 to 63 times (the content of polyphenol in cocoa drink is at least 40 times or more of the content of caffeine content, and specifically 46 to 116 times).

### Example 5

### Study of Caffeine Extraction Efficiency

### (1) Study of Temperature and Pressure

Using, as the cacao raw material, non-alkali treatment cocoa powder (average particle diameter of 32.7 μm) prepared from cocoa cake in the same manner as in Example 1, theobromine and polyphenol retention ratios, and a caffeine reduction ratio by supercritical carbon dioxide extraction treatment at the following temperature and pressure were measured.

### Conditions of Supercritical Fluid Extraction

Apparatus: supercritical fluid extraction system SFX1220 (Model 220), manufactured by Teledyne ISCO, Inc.

Cacao raw material: 0.5 g

Pressure: 10, 20, 30 MPa

Temperature: 50, 70, 90° C.

Solvent: CO₂

Cosolvent: water (45% by weight)

Humidification time: 10 minutes (temperature rise simultaneously)

Extraction time: static extraction for 20 minutes+dynamic extraction for 14 to 48 minutes

Solvent ratio (S/F): 55.6

The concentration of each component, and a caffeine reduction ratio and theobromine and polyphenol retention ratios before and after treatment of supercritical carbon dioxide extraction were calculated in the same manner as in Example 1. The concentration of procyanidins before and after treatment of supercritical carbon dioxide extraction was determined based on a conventional method for measuring procyanidins, and then a procyanidins retention ratio was calculated in the same manner as in theobromine and polyphenol.

The conventional method for measuring procyanidins was performed based on Kelm, M. A. et al., J. Agric. Food. Chem. 2006, 54(5), 1571-1576, and Bergmann, W. R. et al., J. Am. Chem. Soc. 1987, 109, 6614-6619. Specifically, each composition was dissolved in 50% ethanol and then chromatography separation was performed. The chromatography separation was carried out using a HPLC-FL system composed of a binary pump, a solvent degasser, a column oven, an autosampler, a system controller, and a fluorescent detector RF-10A XL (manufactured by Shimadzu Corporation). Analysis was performed by injection (35° C., 5 μl) using a Develosil 100-Diol-5 column having a particle diameter of 5 μm (4.6×250 mm) (NOMURA CHEMICAL CO., LTD.). Two mobile phases are (A) acetonitrile and acetic acid (98:2, v/v), and (B) methanol, water, and acetic acid (95:3:2, v/v). Separation was generated by a series of linear concentration gradient method of the following A from B at a flow ratio of 1 ml/minute:0-60 min, 0-37.6% B in A; 60 minutes, 100% B in A; 60-70 minutes, 100% B in A; 70 minutes, 0% B in A; 70-80 minutes, 0% B in A. Fluorescence detection was recorded at an excitation wavelength of 230 nm and a fluorescent wavelength of 321 nm. The results of the amount of procyanidins were indicated by epicatechin equivalents (average±standard error).

A ratio of the concentration of caffeine to the concentration of the obtained procyanidins was determined and designated as PC/C.

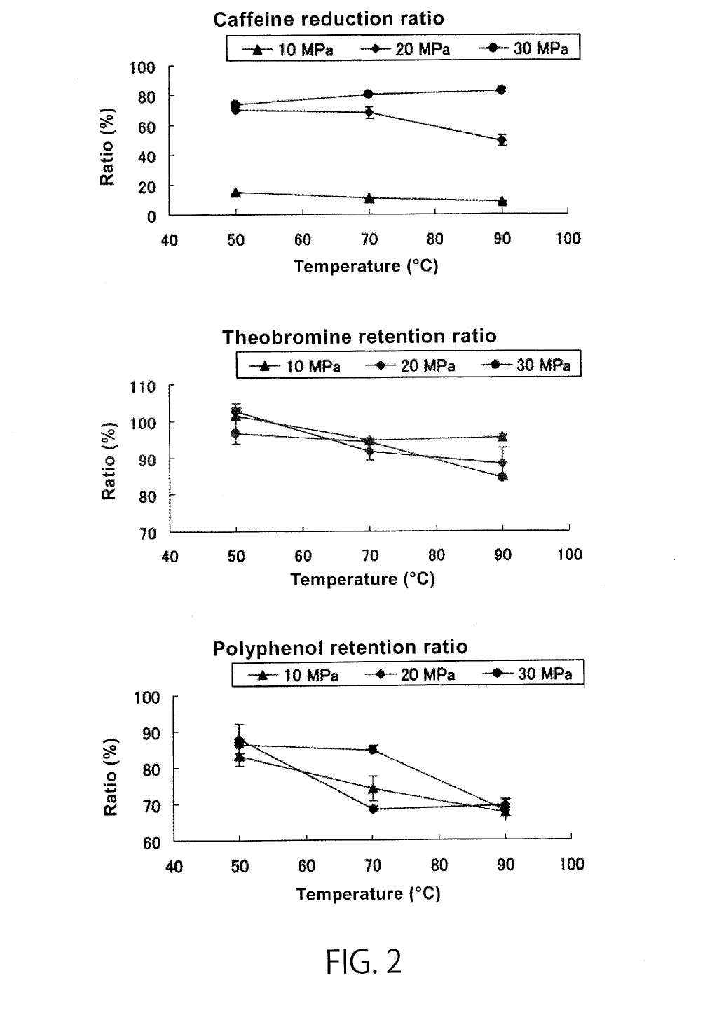

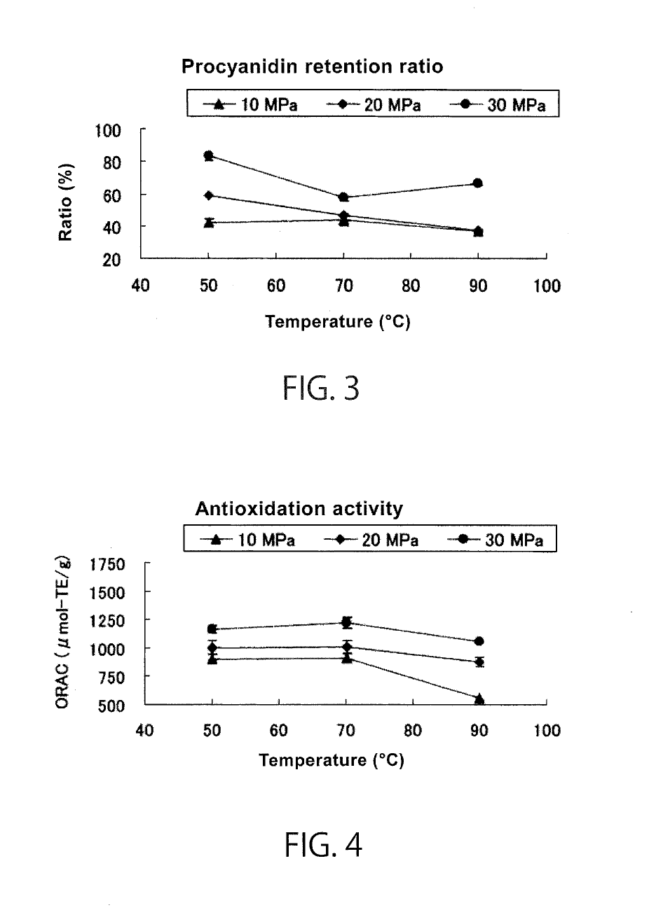

The results of caffeine, theobromine, and polyphenol are shown in Table 7 and FIG. 2. The results of procyanidins are shown in Table 8 and FIG. 3.

TABLE 7
Component (caffeine, theobromine, polyphenol) concentration, and reduction ratio/retention ratio,
component ratio of cacao raw material before and after supercritical carbon dioxide extraction at various
temperatures and pressures

| | | | Caffeine | | | Theobromine | | |
|---|---|---|---|---|---|---|---|---|
| | | Dynamic | Concentration | Concentration | | Concentration | Concentration | |
| | | extraction | before | after | Removal | before | after | |
| Pressure | Temperature | time | treatment | treatment | ratio | treatment | treatment | Retention |
| (MPa) | (° C.) | (minutes) | (mg/g) | (mg/g) | (%) | (mg/g) | (mg/g) | ratio (%) |
| 10 | 50 | 42 | 2.15 ± 0.01 | 1.85 ± 0.01 | 14.91 ± 0.69 | 29.21 ± 0.13 | 29.99 ± 0.13 | 101.41 ± 2.08 |
| | 70 | 45 | 2.15 ± 0.01 | 2.03 ± 0.02 | 10.55 ± 0.15 | 29.21 ± 0.13 | 29.21 ± 0.23 | 94.72 ± 0.34 |
| | 90 | 48 | 2.15 ± 0.01 | 2.02 ± 0.02 | 8.67 ± 0.39 | 29.21 ± 0.13 | 28.70 ± 0.30 | 85.45 ± 0.34 |
| 20 | 50 | 21 | 2.15 ± 0.01 | 0.72 ± 0.02 | 69.89 ± 0.15 | 29.21 ± 0.13 | 33.47 ± 0.35 | 102.51 ± 2.30 |
| | 70 | 23 | 2.15 ± 0.01 | 0.82 ± 0.09 | 68.14 ± 3.81 | 29.21 ± 0.13 | 32.15 ± 0.67 | 91.52 ± 2.34 |
| | 90 | 24 | 2.15 ± 0.01 | 1.18 ± 0.08 | 49.16 ± 3.75 | 29.21 ± 0.13 | 27.77 ± 1.27 | 88.20 ± 4.20 |
| 30 | 50 | 14 | 2.15 ± 0.01 | 0.68 ± 0.02 | 73.52 ± 1.47 | 29.21 ± 0.13 | 33.75 ± 0.00 | 96.66 ± 2.97 |
| | 70 | 15 | 2.15 ± 0.01 | 0.52 ± 0.03 | 80.12 ± 1.05 | 29.21 ± 0.13 | 33.44 ± 0.08 | 94.12 ± 0.02 |
| | 90 | 16 | 2.15 ± 0.01 | 0.45 ± 0.02 | 82.86 ± 1.53 | 29.21 ± 0.13 | 30.15 ± 1.41 | 84.29 ± 0.51 |

| | | Polyphenol | | | | |
|---|---|---|---|---|---|---|
| | | Concentration | Concentration | | | |
| | | before | after | | Component | |
| Pressure | Temperature | treatment | treatment | Retention | ratio | |
| (MPa) | (° C.) | (mg/g) | (mg/g) | ratio (%) | T/C | P/C |
| 10 | 50 | 62.32 ± 1.26 | 52.44 ± 2.45 | 83.05 ± 2.54 | 8.0 | 28.3 |
| | 70 | 62.32 ± 1.26 | 38.73 ± 1.70 | 74.10 ± 3.43 | 14.4 | 24.0 |
| | 90 | 62.32 ± 1.26 | 43.34 ± 1.08 | 67.55 ± 1.24 | 14.2 | 21.5 |
| 20 | 50 | 62.32 ± 1.26 | 61.19 ± 0.72 | 87.92 ± 3.94 | 46.5 | 85.0 |
| | 70 | 62.32 ± 1.26 | 51.39 ± 0.33 | 68.57 ± 0.77 | 39.2 | 62.7 |
| | 90 | 62.32 ± 1.26 | 46.73 ± 1.16 | 69.55 ± 1.60 | 23.5 | 39.6 |
| 30 | 50 | 62.32 ± 1.26 | 64.28 ± 1.09 | 86.25 ± 1.19 | 49.6 | 94.5 |
| | 70 | 62.32 ± 1.26 | 64.17 ± 0.98 | 84.67 ± 1.13 | 64.3 | 123.4 |
| | 90 | 62.32 ± 1.26 | 52.33 ± 0.05 | 68.71 ± 2.87 | 67.0 | 116.3 |

A reduction ratio is entered as for caffeine, and a retention ratio is entered as for theobromine and polyphenol.
T/C represents a ratio of theobromine concentration/caffeine concentration, and P/C represents a ratio of polyphenol concentration/caffeine concentration
n = 3, Average ± SE (standard error)

TABLE 8
Component (procyanidins) concentration, and retention
ratio, component ratio of cacao raw material before and after
supercritical carbon dioxide extraction at various temperatures and
pressures

| | | | Procyanidins | | | |
|---|---|---|---|---|---|---|
| | | Dynamic | Concentration | Concentration | | |
| | | extraction | before | after | Retention | Component |
| Pressure | Temperature | time | treatment | treatment | ratio | ratio |
| (MPa) | (° C.) | (minutes) | (mg/g) | (mg/g) | (%) | PC/C |
| 10 | 50 | 42 | 15.14 ± 0.26 | 6.43 ± 0.28 | 42.24 ± 1.88 | 3.5 |
| | 70 | 45 | 15.14 ± 0.26 | 6.61 ± 0.53 | 43.69 ± 3.52 | 3.3 |
| | 90 | 48 | 15.14 ± 0.26 | 5.64 ± 0.17 | 37.22 ± 1.09 | 2.8 |
| 20 | 50 | 21 | 15.14 ± 0.26 | 8.92 ± 0.07 | 58.92 ± 0.46 | 12.4 |
| | 70 | 23 | 15.14 ± 0.26 | 7.12 ± 0.06 | 47.01 ± 0.39 | 8.7 |
| | 90 | 24 | 15.14 ± 0.26 | 5.66 ± 0.02 | 37.40 ± 0.13 | 4.8 |
| 30 | 50 | 14 | 15.14 ± 0.26 | 12.65 ± 0.39 | 83.56 ± 2.58 | 18.6 |
| | 70 | 15 | 15.14 ± 0.26 | 8.79 ± 0.37 | 58.06 ± 2.43 | 16.9 |
| | 90 | 16 | 15.14 ± 0.26 | 10.11 ± 0.16 | 66.77 ± 1.06 | 22.5 |

A retention ratio is entered as for procyanidins.
P/C represents a ratio of procyanidins concentration/caffeine concentration
n = 3, Average ± SE (standard error)

Furthermore, antioxidation activity of the obtained composition was evaluated. Specifically, each composition was dissolved in 50% ethanol and then the ORAC value was measured. The ORAC value was measured according to a normal method using Spectra Max Gemini (manufactured by MOLECULAR DEVICES CORPORATION, U.S.A) (see Singleton, V. L. et al., Am. J. Enol. Vitic. 1965, 16, 144-158., Takebayashi, J. et al., a preliminary study. Biosci. Biotechnol. Biochem. 2010, 74(10), 2137-2140, and Huang, D. et al., J. Agric. Food. Chem. 2002, 50, 4437-4444). The ORAC value was represented by trolox equivalent (μmol-TE/g) per 1 g of a sample. The results are shown in Table 9 and FIG. 4.

TABLE 9
ORAC value of cacao raw material before and after supercritical carbon
dioxide extraction at various temperatures and pressures

| | | | ORAC value | |
|---|---|---|---|---|
| | | Dynamic | Concentration | Concentration |
| | Temper- | extraction | before | after |
| Pressure | ature | time | treatment | treatment |
| (MPa) | (° C.) | (minutes) | (μmol-TE/g) | (μmol-TE/g) |
| 10 | 50 | 42 | 1428.9 ± 1.11 | 902.6 ± 12.5 |
| | 70 | 45 | 1428.9 ± 1.11 | 911.6 ± 42.3 |
| | 90 | 48 | 1428.9 ± 1.11 | 555.3 ± 21.2 |
| 20 | 50 | 21 | 1428.9 ± 1.11 | 1001.5 ± 59.6 |
| | 70 | 23 | 1428.9 ± 1.11 | 1006.9 ± 55.9 |
| | 90 | 24 | 1428.9 ± 1.11 | 874.5 ± 38.8 |
| 30 | 50 | 14 | 1428.9 ± 1.11 | 1164.2 ± 34.1 |
| | 70 | 15 | 1428.9 ± 1.11 | 1218.5 ± 50.0 |
| | 90 | 16 | 1428.9 ± 1.11 | 1057.1 ± 6.9 |

*n = 3, Average ± SE (standard error)

### (2) Study of Proportion of Cosolvent

Using, as the cacao raw material, non-alkali-treated cocoa powder (average particle diameter of 32.7 μm) prepared from cocoa cake in the same manner as in Example 1, theobromine and polyphenol retention ratios, and a caffeine reduction ratio by a treatment of supercritical carbon dioxide extraction (SFE) at proportions of the following cosolvent were measured.

### Conditions of Supercritical Fluid Extraction

Apparatus: supercritical fluid extraction system SFX1220 (Model 220), manufactured by Teledyne ISCO, Inc.

Cacao raw material: 0.5 g

Pressure: 30 MPa

Temperature: 70° C.

Solvent: CO₂

Cosolvent: water (0, 15, 30, 45, 60, 75% by weight)

Humidification time: 10 minutes (temperature rise simultaneously)

Extraction time: static extraction for 20 minutes+dynamic extraction for 15 minutes

Solvent ratio (S/F): 55.6

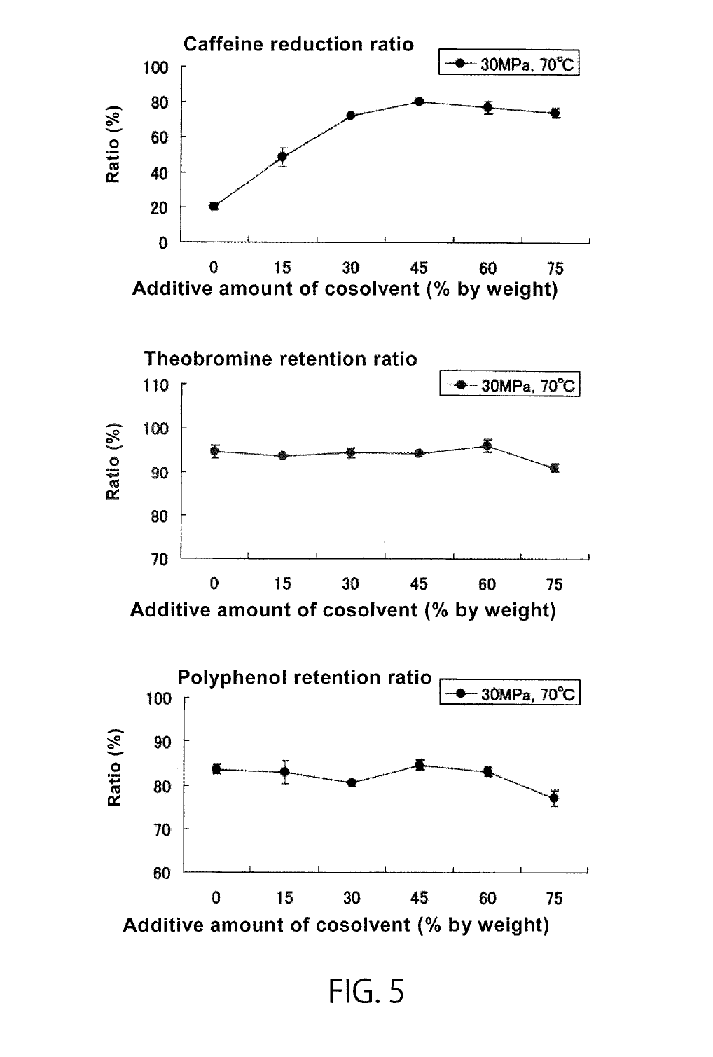

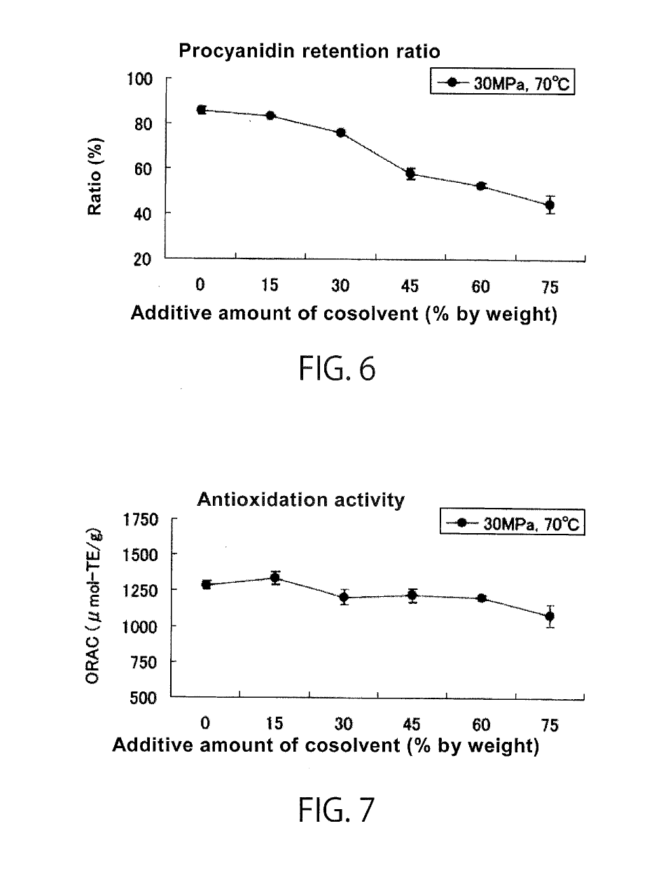

The concentration of each component, and a caffeine reduction ratio and theobromine and polyphenol retention ratios before and after treatment of supercritical carbon dioxide extraction were calculated in the same manner as in Example 1. The results are shown in Table 10 and FIG. 5. The concentration of procyanidins and a procyanidins retention ratio before and after treatment of supercritical carbon dioxide extraction were calculated in the same manner as in Example 5(1). The results are shown in Table 11 and FIG. 6.

TABLE 10
Component concentration, and reduction ratio/retention ratio, component ratio of cacao raw
material before and after supercritical carbon dioxide extraction at various proportions of cosolvent

| | Caffeine | | | Theobromine | | |
|---|---|---|---|---|---|---|
| Cosolvent | Concentration | Concentration | | Concentration | Concentration | |
| Proportion | before | after | Removal | before | after | |
| (% by | treatment | treatment | ratio | treatment | treatment | Retention |
| weight) | (mg/g) | (mg/g) | (%) | (mg/g) | (mg/g) | ratio (%) |
| 0 | 2.15 ± 0.01 | 2.13 ± 0.05 | 20.07 ± 1.79 | 29.21 ± 0.13 | 34.24 ± 0.42 | 94.56 ± 1.37 |
| 15 | 2.15 ± 0.01 | 1.43 ± 0.12 | 48.26 ± 5.47 | 29.21 ± 0.13 | 35.08 ± 0.65 | 93.52 ± 0.16 |
| 30 | 2.15 ± 0.01 | 0.74 ± 0.01 | 71.89 ± 0.15 | 29.21 ± 0.13 | 33.50 ± 0.28 | 94.32 ± 1.12 |
| 45 | 2.15 ± 0.01 | 0.52 ± 0.09 | 80.12 ± 1.05 | 29.21 ± 0.13 | 33.44 ± 0.08 | 94.12 ± 0.02 |
| 60 | 2.15 ± 0.01 | 0.58 ± 0.09 | 77.10 ± 3.55 | 29.21 ± 0.13 | 33.00 ± 0.51 | 96.02 ± 1.49 |
| 75 | 2.15 ± 0.01 | 0.66 ± 0.06 | 74.03 ± 2.50 | 29.21 ± 0.13 | 31.44 ± 0.13 | 90.98 ± 0.85 |

| | Polyphenol | | | | |
|---|---|---|---|---|---|
| Cosolvent | Concentration | Concentration | | | |
| Proportion | before | after | | Component | |
| (% by | treatment | treatment | Retention | ratio | |
| weight) | (mg/g) | (mg/g) | ratio (%) | T/C | P/C |
| 0 | 62.32 ± 1.26 | 64.61 ± 0.68 | 83.63 ± 1.06 | 16.1 | 30.3 |
| 15 | 62.32 ± 1.26 | 66.36 ± 0.79 | 82.98 ± 2.67 | 24.5 | 46.4 |
| 30 | 62.32 ± 1.26 | 60.97 ± 0.77 | 80.46 ± 0.75 | 45.3 | 82.4 |
| 45 | 62.32 ± 1.26 | 64.17 ± 0.98 | 84.67 ± 1.13 | 64.3 | 123.4 |
| 60 | 62.32 ± 1.26 | 60.21 ± 0.73 | 83.12 ± 1.00 | 56.9 | 103.8 |
| 75 | 62.32 ± 1.26 | 56.77 ± 1.08 | 77.01 ± 1.86 | 47.6 | 86.0 |

A reduction ratio is entered as for caffeine, and a retention ratio is entered as for theobromine and polyphenol.
T/C represents a ratio of theobromine concentration/caffeine concentration, and P/C represents a ratio of polyphenol concentration/caffeine concentration
n = 3, Average ± SE (standard error)

TABLE 11
Component (procyanidins) concentration, and retention ratio, component
ratio of cacao raw material before and after supercritical carbon dioxide
extraction at various proportions of cosolvent

| | Procyanidins | | | |
|---|---|---|---|---|
| Cosolvent | Concentration | Concentration | | |
| Proportion | before | after | | Component |
| (% by | treatment | treatment | Retention | ratio |
| weight) | (mg/g) | (mg/g) | ratio (%) | PC/C |
| 0 | 15.14 ± 0.26 | 12.96 ± 0.24 | 85.58 ± 1.60 | 6.1 |
| 15 | 15.14 ± 0.26 | 12.64 ± 0.04 | 83.47 ± 0.26 | 8.8 |
| 30 | 15.14 ± 0.26 | 11.49 ± 0.16 | 75.92 ± 1.04 | 15.5 |
| 45 | 15.14 ± 0.26 | 8.79 ± 0.37 | 58.06 ± 2.43 | 16.9 |
| 60 | 15.14 ± 0.26 | 7.99 ± 0.15 | 52.76 ± 0.97 | 13.8 |
| 75 | 15.14 ± 0.26 | 6.76 ± 0.61 | 44.66 ± 4.02 | 10.2 |

*A retention ratio is entered as for procyanidins.
*P/C represents a ratio of procyanidins concentration/caffeine concentration
*n = 3, Average ± SE (standard error)

Furthermore, antioxidation activity of the obtained composition was evaluated in the same manner as in Example 5(1). The results are shown in Table 12 and FIG. 7.

TABLE 12
ORAC value of cacao raw material before and after supercritical
carbon dioxide extraction at various proportions of cosolvent

| | ORAC value | |
|---|---|---|
| | Concentration | Concentration |
| Cosolvent | before | after |
| Proportion | treatment | treatment |
| (% by weight) | (μmol-TE/g) | (μmol-TE/g) |
| 0 | 1428.9 ± 11.1 | 1287.4 ± 26.3 |
| 15 | 1428.9 ± 11.1 | 1335.3 ± 4.29 |
| 30 | 1428.9 ± 11.1 | 1203.0 ± 53.1 |
| 45 | 1428.9 ± 11.1 | 1218.5 ± 50.0 |
| 60 | 1428.9 ± 11.1 | 1203.8 ± 14.6 |
| 75 | 1428.9 ± 11.1 | 1079.5 ± 75.9 |

*n = 3, Average ± SE (standard error)

(3) Correlation between Total Polyphenol Concentration or Procyanidins Concentration and Antioxidation Activity

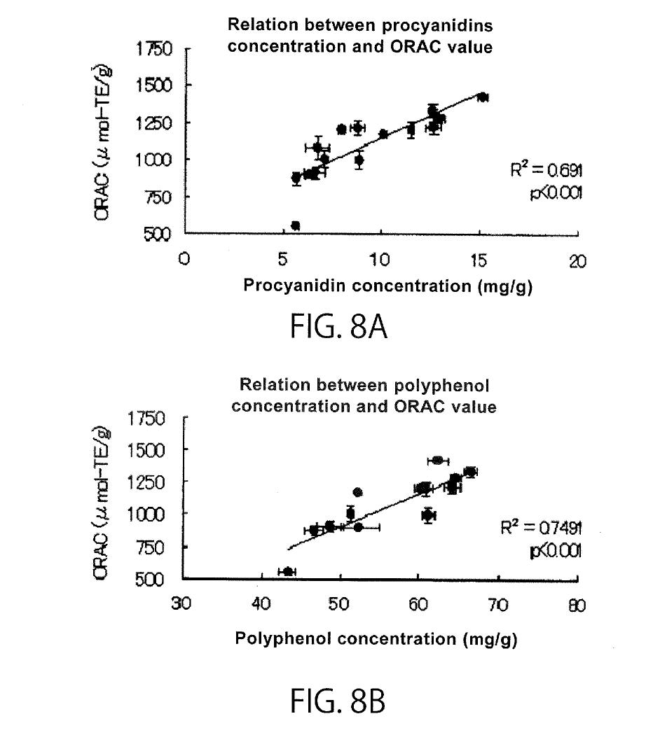

A correlation between the total polyphenol concentration or procyanidins concentration before and after treatment of supercritical carbon dioxide extraction, and the ORAC value was analyzed using a regression line of data obtained by calculation of the Pearson correlation coefficient (r) at reliability of 95%. Regarding statistical significance, the case where two-sided p value is less than 5% was judged as significant results. The results are shown in FIG. 8.

As shown in the results, positive correlation between antioxidation activity before treatment and procyanidins was recognized (r=0.83, p<0.001), and positive correlation between antioxidation activity after treatment and total polyphenol was recognized (r=0.87, p<0.001). In other words, it is considered that antioxidation activity of the cacao raw material depends on the total polyphenol concentration. Therefore, in the caffeine-reduced cacao composition of the present invention, even when the concentration of procyanidins, which is said to be a main factor of antioxidation activity, decreases, antioxidation activity does not decrease as far as the total polyphenol concentration is retained to some extent.

### (4) Study of Solvent Ratio

Using, as the cacao raw material, non-alkali-treated cocoa powder (average particle diameter of 32.7 μm) prepared from cocoa cake in the same manner as in Example 1, theobromine and polyphenol retention ratios, and a caffeine reduction ratio by a treatment of supercritical carbon dioxide extraction at the following solvent ratio were measured.

### Conditions of Supercritical Fluid Extraction

Apparatus: NOVA 300 ml TWIN, manufactured by NOVA SWISS

Cacao raw material: 75.5 g

Pressure: 30 MPa

Temperature: 70° C.

Solvent: CO₂

Cosolvent: water (45% by weight)

Humidification time: 10 minutes (temperature rise simultaneously)

Extraction time: static extraction for 20 minutes+dynamic extraction for 29 to 688 minutes

Solvent ratio (S/F): 7.5, 15.0, 32.5, 47.5, 62.5, 80.0, 95.0, 120.0, 150.0, 180.0

The concentration of each component, and a caffeine reduction ratio and theobromine and polyphenol retention ratios before and after treatment of supercritical carbon dioxide extraction were calculated in the same manner as in Example 1.

The results of the obtained caffeine reduction ratio, and theobromine and polyphenol retention ratios were respectively plotted against a solvent ratio to make a graph (not shown), and then an optimum value of a solvent ratio was studied. The results revealed that the solvent ratio for obtaining a desired caffeine-reduced cacao composition is desirably 55.0 or more, and preferably 94.0 or more. Low solvent ratio leads to poor caffeine removal ratio (extraction ratio). If the extraction ratio reached 120.0, the amount of caffeine extracted and removed from the extraction raw material reached a saturated state. The theobromine retention ratio scarcely changed under the conditions of this SFE treatment. The polyphenol retention ratio slightly decreased as the solvent ratio increases, but the retention ratio did not reach 88% by weight or less.

1. A caffeine-reduced cacao composition obtained by subjecting at least one cacao raw material selected from cacao mass and cocoa powder to supercritical carbon dioxide extraction using water as a cosolvent, wherein
the concentration of theobromine in the composition is 0.7% by weight or more, and
the content of theobromine in the composition is 25 times or more of the content of caffeine.

2. The caffeine-reduced cacao composition according to claim 1, wherein the content of polyphenol in the caffeine-reduced cacao composition is 40 times or more of the content of caffeine.

3. The caffeine-reduced cacao composition according to claim 1, wherein the cacao raw material is not subjected to an alkali treatment.

4. The caffeine-reduced cacao composition according to claim 1, wherein the cacao raw material is cocoa powder.

5. The caffeine-reduced cacao composition according to claim 1, wherein supercritical carbon dioxide extraction is performed under the conditions of 20 MPa or more and 50° C. or higher.

6. The caffeine-reduced cacao composition according to claim 1, wherein the proportion of the cosolvent relative to the cacao raw material in supercritical carbon dioxide extraction is 30% by weight or more.

7. The caffeine-reduced cacao composition according to claim 1, wherein 90% by weight or more of the content of theobromine derived from the cacao raw material is retained, and also 60% by weight or more of the content of caffeine derived from the cacao raw material is reduced.

8. The caffeine-reduced cacao composition according to claim 1, wherein 70% by weight or more of the content of polyphenol derived from the cacao raw material is retained.

9. A food and drink composition comprising the caffeine-reduced cacao composition according to claim 1.

10. A method for producing a caffeine-reduced cacao composition, which comprises subjecting at least one cacao raw material selected from cacao mass and cocoa powder to supercritical carbon dioxide extraction using water as a cosolvent to obtain a cacao composition in which theobromine derived from the cacao raw material and, if necessary, polyphenol is retained, and caffeine derived from the cacao raw material is reduced.

11. The method for producing a caffeine-reduced cacao composition according to claim 10, wherein the content of theobromine in the obtained caffeine-reduced cacao composition is 25 times or more of the content of caffeine.

12. The method for producing a caffeine-reduced cacao composition according to claim 10, wherein the content of polyphenol in the obtained caffeine-reduced cacao composition is 40 times or more of the content of caffeine.

13. The method for producing a caffeine-reduced cacao composition according to claim 10, wherein supercritical carbon dioxide extraction is performed under the conditions of 20 MPa or more and 50° C. or higher.

14. The method for producing a caffeine-reduced cacao composition according to claim 10, wherein the proportion of the cosolvent relative to the cacao raw material in supercritical carbon dioxide extraction is 30% by weight or more, and the cacao raw material is humidified by adding the cosolvent, and then the supercritical carbon dioxide extraction is performed.

15. The method for producing a caffeine-reduced cacao composition according to claim 10, which comprises performing static extraction by keeping a pressure-up state in supercritical carbon dioxide extraction.

16. The method for producing a caffeine-reduced cacao composition according to claim 10, wherein a solvent ratio of the weight of the solvent composed of carbon dioxide to the weight of the cacao raw material is 55.0 or more.

17. The method for producing a caffeine-reduced cacao composition according to claim 10, wherein 90% by weight or more of the content of theobromine derived from the cacao raw material in the obtained caffeine-reduced cacao composition is retained, and also 60% by weight or more of the content of caffeine derived from the cacao raw material in the obtained caffeine-reduced cacao composition is reduced.

18. The method for producing a caffeine-reduced cacao composition according to claim 10, wherein 70% by weight or more of the content of polyphenol derived from the cacao raw material in the obtained caffeine-reduced cacao composition is retained.

19. A method for retaining the content of theobromine derived from the cacao raw material 90% by weight or more and, if necessary, for retaining the content of polyphenol derived from the cacao raw material 70% by weight or more and also for reducing the content of caffeine derived from the cacao raw material 60% by weight or more, comprising subjecting a cacao raw material to supercritical carbon dioxide extraction using water as a cosolvent.
